(19) United States
(12) Patent Application Publication
(10) Pub. No.: US 2008/0199914 A1
(43) Pub. Date: Aug. 21, 2008

(54) Modified Alginates, Methods of Production and Use

(75) Inventors: Gudmund Skjak-Braek, Trondheim (NO); Ivan Donati, Udine (IT)

Correspondence Address: Patent Administrator;FMC CORPORATION, 1735 Market Street, Philadelphia, PA, 19103, US

(73) Assignee: FMC BIOPOLYMER AS, Drammen (NO)

(21) Appl. No.: 11/719,076

(22) PCT Filed: Nov. 11, 2005
(86) PCT No.: PCT/IB2005/003633

§ 371 Date: Dec. 14, 2007

Related U.S. Application Data
(60) Provisional application No. 60/627,057, filed on Nov. 12, 2004.
(60) Provisional application No. 60/627,247, filed on Nov. 12, 2004.
(60) Provisional application No. 60/630,867, filed on Nov. 24, 2004.

(51) Int. Cl. C12P 19/26; C12N 11/10

(52) U.S. Cl. 435/84; 435/178

(57) ABSTRACT

Process for preparing a modified alginate polymers are disclosed. The processes comprise the steps of covalently attaching a modifying moiety to one or more unmodified monomeric subunits of an alginate polymer; and changing one or more unmodified mannuronic (M) monomeric subunits of the alginate polymer to one or more unmodified guluronic (G) monomeric sub-units by an enzymatic epimerization reaction performed in any order. Processes for preparing alginate gels, fiber, and compositions are also disclosed. Modified alginates in which only M monomeric subunits are modified, and alginate gels, fibers and compositions comprising the same, are disclosed.

This application claims priority to U.S. Provisional Application No. 60627057 filed Nov. 12, 2004, U.S. Provisional Application No. 60/627,247 filed Nov. 12, 2004, and U.S. Provisional Application No. 60/630,867 filed Nov. 24, 2004, which are each incorporated herein by reference.

### FIELD OF THE INVENTION

The present invention relates to modified alginates prepared by a chemoenzymatic modification of alginate polymers as described herein, methods of preparation and uses thereof.

### BACKGROUND OF THE INVENTION

Chemically, alginates are linear copolymers of 1→4 linked β-D-mannuronic acid (M) and α-L-guluronic acid (G) arranged in a blockwise pattern along the chain with homopolymeric regions of M (M-blocks) and G (G-blocks) residues interspersed with regions of alternating structure (MG-blocks). In nature, alginates are produced first as homopolymeric mannuronan and converted to heteropolymers contains M and (monomers subunits via a post-polymerization epimerization reaction involving a C-5 inversion on the M residues of mannuronan. This reaction is catalyzed by the mannuronan C-5 epimerases.

Recently, it has been found that the genome of the alginate-producing bacterium Azotobacter vinelandii encodes seven different mannuronan C-5-epimerase genes. These genes have been sequenced, cloned and expressed in Escherichia coli; the enzymes thus produced have been designated AlgE1-AlgE7. Since all natural alginates are produced from homopolymeric mannuronan by the same basic C-5 inversion from M to G, the remarkable variability in composition and sequence found in the polysaccaride is solely due to the different catalytic properties of the different epimerases. As an example, while AlgE4 predominantly forms alginates with MG-blocks, AlgE6 introduces long G-blocks into the polymer. The availability of these alginate-modifying enzymes and their use makes it possible to produce alginates with tailored structural and physical properties.

Alginates form cross-linked gels in the presence of divalent cations which cross link G monomer subunits of polymers with ionic bonds. The rapid gel formation of alginate, in the presence of millimolar concentrations of calcium, depends on the fraction of G residues as well as on the sequence pattern of G and M residues.

In the last decade there has been an increasing interest in the use of alginates in increasingly demanding end uses such as biotechnological, biomedical and pharmaceutical applications. The use of alginate as immobilisation material for cells and biocatalysts is an example of this trend. The possible use of such systems in industry, medicine and agriculture are numerous and range from production of ethanol from yeast and monoclonal antibodies from hybridomas, to mass production of artificial seeds by entrapment of plant embryos.

Alginate gels also have potential as Extracellular matrix material (ECM) for cell immobilisation, transplantation and tissue engineering. However, in spite of the interesting physical and mass transport aspects of calcium-alginate hydrogels; their application is limited due to biological inertness (e.g. cell adhesion and signalling). Although alginate entrapment is a very gentle technique for immobilising living cells, many cells need specific interaction with the matrix for their proliferation and viability. Such anchoring dependent behaviour is common for most mammalian cells; however the alginate network itself is non-interacting.

Since alginate is known to be a non bioadhesive material, the introduction of cell-specific ligands or extracellular signalling molecules, such as peptides or oligosaccharides, is necessary for its direct involvement in the cell-cell and cell-ECM recognition processes. Along this line, third-generation biomaterials based on such modified alginates have already been reported to be able to significantly enhance the interaction with cells, disclosing new opportunities and future development in the field of polymer engineering and tissue regeneration. However, the design of an adequate ECM-mimicking scaffold relies, beside fundamental biological aspect, also on physical properties such as gel formation, mechanical strength and stability.

The ionotropic gelation properties have established alginate as an appealing candidate for biotechnological and medical applications, in particular in the field of cell and tissue encapsulations. As an example, alginate-poly-L-lysine capsules containing pancreatic islets of Langerhans have been shown to reverse diabetes in large animals, where the stable and selectively permeable barrier represented by the capsule protects the transplanted cells from the immune system of the host.

Various ligands have been coupled to alginate polymers to improve cell/matrix interaction, as in U.S. Pat. No. 6,642,362 issued Nov. 4, 2003 to Mooney et al. A major problem with chemical modification of alginates is that such modification is often not chemoselective. That is, modifications can occur on both saccharide monomers (guluronic acid (G) and mannuronic acid (M)) of which alginate is comprised. It is further known that gel formation, and in particular gel strength, is a property related to the number of unmodified G's. Chemical modification of alginates described in the prior art describe substitution that is not restricted to the M residues (M units) but also take place in the G-residues (G units) in the G-blocks thereby reducing the amount of available G residues thus impairing the co-operative binding of divalent cations and decreasing rate of gel formation which results in weak gel format ion and uncontrollable swelling in saline. As used herein, a “residue” refers to a single M or G unit and a “block” refers to multiple units of M, G or MG.

The synthesis and characterization of a galactose-substituted alginate, obtained by introducing 1-amino-1-deoxy-β-galactose residues on the uronic groups of the polysaccharide chain has been reported. Based on the recognition of β-galactose moieties by the ASialoGlycoProtein Receptor (ASGP-R) present on the cell surface of hepatocytes and considering reported results, modified alginates have been proposed as suitable gel-forming biomaterial to improve encapsulation and adhesion of hepatocytes. However, the characterization of the modified alginate at a molecular level revealed that the introduction of the side-chain groups on alginate chain mainly affects the G residues, therefore impairing the calcium-binding properties and, as a consequence, leading to less stable hydrogels. A considerable decrease in rigidity and stability of the modified calcium-alginate hydrogels have already been reported. It therefore appears that the introduction of cell-specific ligands on the polysaccharide chain may lead to a drop in mechanical properties of the hydrogel.

In this perspective, a considerable improvement would be represented by the production of a selectively modified alginate bearing side chain molecules on mannuronic residues, in order to limit the calcium binding impairment and the loss of stability of the hydrogel. Similarly, there is a need to improve the properties of modified alginate that bear side chain molecules, in order to limit the cation binding impairment and the loss of stability of the hydrogel The present invention enables control over mechanical and swelling properties of alginate gels after substitution with various ligands as explained below.

### SUMMARY OF THE INVENTION

The present invention relates to processes for preparing a modified alginate polymer. The processes comprise the steps of covalently attaching a modifying moiety to one or more unmodified monomeric subunits of an alginate polymer, and changing one or more unmodified mannuronic (M) monomeric subunits of the alginate polymer to one or more unmodified guluronic (G) monomeric subunits by an enzymatic epimerization reaction. The reaction steps may be preformed in either order and multiple times in any sequence.

The present invention further relates to processes for preparing alginate gel and fibers. The processes comprise the step of combining, in a solvent, a plurality of modified alginate polymers with a divalent gelling ion. In some embodiments, living cells are encapsulated within alginate gels.

The present invention relates to modified alginate polymers in which only M monomeric subunits are modified, wherein the modification is not acetylation.

The present invention relates to alginate gels and fibers comprising modified alginate polymers in which only M monomeric subunits are modified, wherein the modification is not acetylation.

The present invention further relates to processes of preparing a shaped or unshaped solid non-crosslinked alginate composition.

### BRIEF DESCRIPTION OF THE FIGURES

FIG. 1 illustrates an example a two-step process for selective substitution of the ManA residues in alginates. First step is a substitution of mannuronan with galactosamine. The second step is a C-5 epimerisation using recombinant produced C-5 epimerase. Example 1 refers to the process shown in FIG. 1.

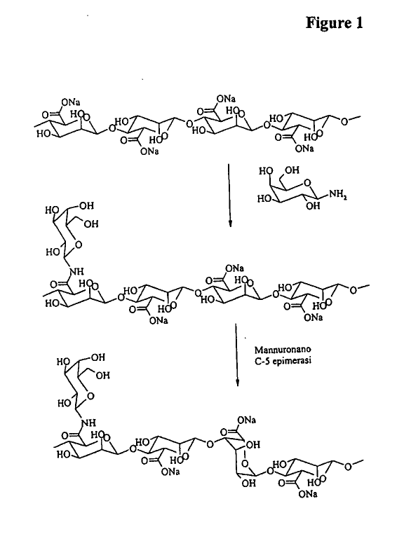

FIG. 2: Swelling of calcium alginate gel beads made from: Squares: L. Hyperborea; Circles: Polymannuronan modified and epimerised (12% of galactose); Triangles: modified L. hyperborea (14% of galactose) with number of changes of saline solution (NaCl 0.9%)

FIG. 3: Mechanical strength measured as Youngs modulus for: 1: unmodified L. hyperborea; 2: modified L. hyperborea (14% of galactose) 3: Polymannuronan modified and epimerised (12% of galactose).

FIG. 4: Effect of selective modification on M residues on swelling of Ca alginate gel beads made from: Modified alginate from Laminaria hyperborea (◯), modified and epimerized mannuronan(□), and L. hyp. alginate (Δ), with number of changes of saline solution.(NaCl 0.9%)

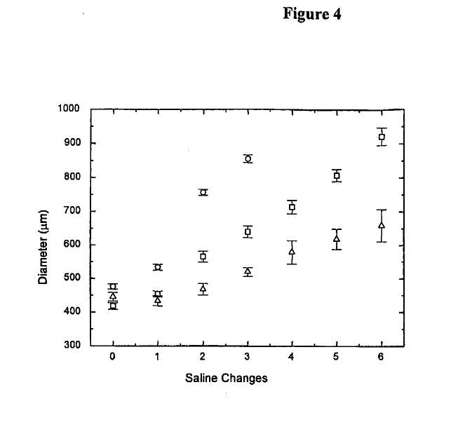

FIG. 5: Effect of photocrosslinking on M-substituted alginate capsules on stability in 50 mM EDTA (A) and swelling in 0.9% NaCl (B) solution, uncrosslinked sample (□), photocrosslinked sample (◯).

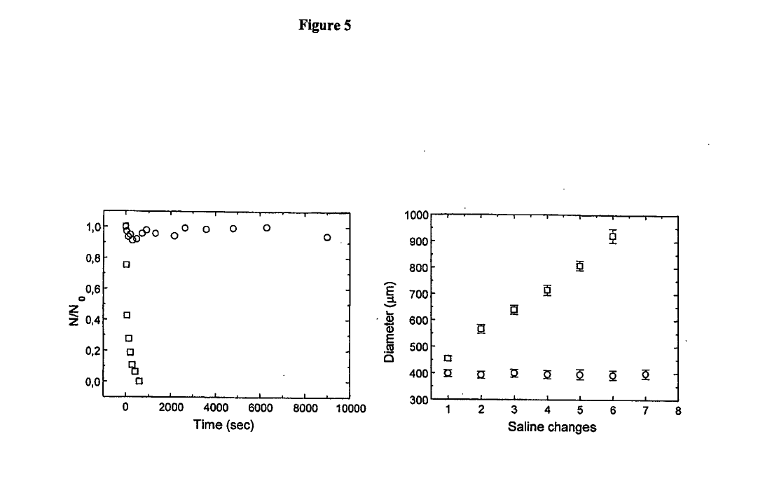

FIG. 6: 300 MHz ¹H-NMR (spectra (anomeric region) of MGal, MGalE4 and MGalE4E6. H1-G represents the anomeric signal of guluronic residues introduced, H5-G(G) represents the H5 signal of a guluronic residue neighboring another guluronic moiety.

FIG. 7: a) Comparison of the efficiency (40) of the epimerase AlgE4 on mannuronan and on MGal sample with respect to the introduction of single G residues in the polymer chain. b) Comparison of the efficiency (%) of the epimerase AlgE6 on polyalternating MG²⁰ (F_{G}=0.47) and on MGalE4 sample with respect to the introduction of single G residues (light gray) and GG diads (dark grey) in the polymer chain.

FIG. 8: 300 MHz ¹H-NMR spectra of a) mannuronan modified with pNH₂PhβGal (d.s.=0.18) and epimerized with) AlgE4 (Final polymer composition. F_{G}=0.26; F_{GG}=0 and then with c) AlgE6 (Final polymer composition: F_{G}=0.36; F_{GG}=0.17).

FIG. 9: Circular dichroism spectra of a) MGal, b) MGalE4 and c) MGalE4E6 before (—) and after (- -) addition of calcium ([Ca²⁺]/[Polym]=0.26 for all the samples reported).

FIG. 10: Variation of a) G′ and b) δ in the first 1000 seconds for gels obtained from samples MaIE4E6 (triangle), LhypCal (circles) and alginate from L. hyperborea (squares), c) Variation of G′ during the curing of the calcium-gels for MGaIM E6 (—), LhypGal (- -) and alginate from L. hyperborea ( . . . ) Gels obtained from a 1.5% polymer solution added of 20 mM CaCO₃ and 40 mM of GDL.

FIG. 11: Storage G′ (solid symbols) and loss G″ (open symbols) moduli for hydrogels obtained from L. hyperborea alginate (squares), LhypGal (circles) and MGal 4E6 (triangles). Gels obtained from a 1.5% polymer solution added of 20 mM CaCO₃ and 40 mM of GDL.

FIG. 12: a) Young's modulus (E) of gel cylinders obtained from L. hyperborea, LhypGal and MGalE4E6. The molar ratio [Ca²⁺]/[G residues] was equal to 0.59 for all the three samples. Values are reported as means ±s.d. (n=8). b) Dependence of the syneresis on the ratio [Ca²⁺]/[Polym] for gel cylinders obtained from L. hyperborea (squares), Lhypal (triangles) and MGalE4E6 (circles). Values are reported as mean ±s.d. (n=8).

FIG. 13: Stability of calcium beads expressed as increase of the absolute diameter (d₀=initial diameter of the bead) for increasing changes of saline solution for alginate from L. hyperborea (squares), LhypGal (triangles) and MGalE4E6 (circles). Values are reported as mean ±s.d.

FIG. 14: Chemoenzymatic approach for the production of alginate selectively modified on M residues. S=1-amino-1-deoxy-β-D-galactose or pNH₂Ph-β-D-galactopyranoside

### DETAILED DESCRIPTION OF PREFERRED EMBODIMENTS

Alginate is a collective term for a family of linear copolymers of D-mannuronic acid and L-guluronic acid in various proportion and sequential arrangements. The ability of alginate polymers to form a gel with divalent cations such as calcium, and properties of the resulting gel are strongly correlated with the proportion and length of the blocks of contiguous G residues in the polymer chain.

The present invention provides processes for modifying alginates that require at least two steps: one step in which a modifying moiety is covalently attached to one or more unmodified monomeric subunits of an alginate polymer and another step in which one or more unmodified mannuronic (M) monomeric subunits of the alginate polymer is converted to one or more unmodified guluronic (G) monomeric subunits by an enzymatic epimerization reaction. According to processes of the invention, these steps can be performed in either order. Further, multiple steps in which a modifying moiety is covalently attached to one or more unmodified monomeric subunits of an alginate polymer can be performed and multiple steps in which one or more unmodified mannuronic (M) monomeric subunits of the alginate polymer is converted to one or more unmodified guluronic (G) monomeric subunits by an enzymatic epimerization reaction can be performed. The multiple steps can be performed in any sequence. Monomeric subunits may be modified at either carboxylic groups and hydroxyl groups.

Substitution of functional groups in the alginate will depending on the chemical character and the bulkiness of the constituents, reduce the gel forming capacity of the polymer. This effect can be minimised by increasing the amount G blocks. In some preferred embodiments, substitution of functional groups is limited to substitution of M residues by using alginates with M only as a starting material for modification. After modification, unmodified Ms are converted to G by epimerization.

Modified alginate polymers in which only M monomeric subunits are modified are produced. The modified alginate polymers may comprise unmodified Ms and unmodified: Gs, The modification is not acetylation although some Ms may be acetylated. That is, some of the M monomeric subunits of such a polymer can be modified by a modification other than acetylation whether or not other M monomeric subunits of such a polymer are acetylated. In some preferred embodiments, the modified alginate polymers in which only M monomeric subunits are modified are modified by addition of a modifying moiety such as galactose and oligomers thereof, mannose and oligomers thereof, ste^{x} (NeuAcα2-3Galβ1-[4Fucα1-3]GCNAc), GlcNAc, HA-oligomers (hyaladhesins; hyaluronan binding proteins), RDG peptides, YIGSR peptides, REDV peptides, IKVAV peptides, KHIFSDDSSE peptides, and KRSR peptides. Modified alginate polymers in which only M monomeric subunits are useful to make alginate gels and fibers.

The starting alginate can have varying amounts of M and C which may be grouped in varying structural arrangements of MM, GG, and/or MC blocks. The chemical reaction step will lead to substituents reacted on the M and G residues (modified M residues and modified G residues) of the alginate as applicable. The enzymatic step will change the amount of M and G in the alginate by converting a desired number of M residues to G residues. For example the amount of G is increased by converting MM blocks to MG or GG or converting MG blocks to GG.

In some embodiments, alginates having a high M content are useful such as an M content of at least 50%, 60%, 70%, 80%, 90%, 95%, or 95+%, by total weight of the M and G content. One embodiment of the invention utilizes a homopolymer of mannuronic acid, e.g., a mannuronan, as the starting alginate rich in M residues prior to chemical reaction. These homopolymers can be produced for example by AlgG negative mutants of Pseudomonas aeruginosa, P. syringae or P. fluorescens disclosed in WO 04011628 published Feb. 5, 2004 hereby incorporated by reference. Other examples of high M alginates are disclosed in WO03046199A2 which is incorporated herein by reference.

According to the invention, a modifying moiety can be any chemical structure but is preferable selected from the group consisting of, a monosaccharide, an oligosaccharide, a mononucleotide, an oligonucleotide, an amino acid, a peptide and a protein. In some embodiments, the modifying moiety is selected from the group of those listed in U.S. Pat. No. 6,642,362. In some embodiments, the modifying moiety contains a carbon-carbon double bond or triple bond capable of free radical polymerization. Monosaccharides may be, for example, lactose, galactose, sucrose, fructose, mannose, and cellulose. Oligosaccharides may be homopolymers or heteropolymers made up of monosaccharides such as, for example, lactose, galactose, sucrose, fructose, mannose, and cellulose. Oligosaccharides preferable have 2-10 monomers; more preferably 2-3. Mononucleotides may be for example adenine, guanine, cytosine, thymidine or uracil. An oligonucleotide may be homopolymers or heteropolymers made up of mononucleotides may be for example adenine, guanine, cytosine, thymidine or uracil. Oligonucleotides preferable have 2-150 monomers, more preferably 2-50 monomers, more preferably 5-35 monomers and more preferably 10-20 monomers. Amino acids may be any of the twenty six naturally occurring amino acids as well as any synthetic amino acid residue. Peptides may be homopolymers such as for example poly-lysine or heteropolymers. Peptides preferable have 2-25 monomers, more preferably 2-20 monomers, more preferably 2-15 monomers, more preferably 2-10 monomers, more preferably 2-5 monomers, and more preferably 2, 3 or 4 monomers. Proteins may be any proteinaceous molecules such as cell attachment or adhesion molecules, receptor proteins or ligands. Proteins preferable have greater than 25 amino acids and in some embodiments may be 25-200 amino acids or larger.

In some embodiments, the modifying moiety is a galactose based oligosaccharide such as one which binds to ASGPR asialoglycoprotein receptor or galectin. ASPGR is a hepatocyte adhesion receptor. Galectin is a cell adhesion receptor. In some embodiments, the modifying moiety is sLe^{x} (NeuAca2-3Galβ1-[4Fucα1-3]GlcNAc) which is sectine, a cell-cell recognition molecule. In some embodiments, the modifying moiety is a GlcNAc which is ASGP, also useful as in hepatocyte adhesion. In some embodiments, the modifying moiety is HA-oligomers (hyaladhesins; hyaluronan binding proteins) useful in endothelial cell proliferation. In some embodiments, the modifying moiety is a mannose based oligosaccharide such as one that binds to mannose binding lectine or Langerin. Mannose binding lectine is involved in keratinocyte proliferation. Langerin is a receptor in Langerhans cells.

In some embodiments, the modifying moiety may be an RDG peptide such as those derived from fibronectin or vitronectin. RDG peptide may be useful as a cell adhesion and myoblast adhesion peptides. In some embodiments, the modifying moiety may be a YIGSR peptide such as those derived from laminin B1. YIGSR peptide may be useful as a cell adhesion peptide. In some embodiments, the modifying moiety may be an REDV peptide such as those derived from fibronectin. REDV peptide may be useful as an endothelial cell adhesion peptides. In some embodiments, the modifying moiety may be an IKVAV peptide such as those derived from laminin. IKVAV peptide may be useful as a neurite extension peptides. In some embodiments, the modifying moiety may be an KHIFSDDSSE peptide such as those derived from neural cell adhesion molecules. KHIFSDDSSE peptide and fragments thereof having 2, 3, 4 or more amino acids may be useful as astrocyte adhesion peptides. In some embodiments, the modifying moiety may be an KRSR peptide such as those derived from heparin binding domain. KRSR peptide and may be useful as osteoblast adhesion peptides.

Alginate polymers may be crosslinked by bonds between modifying moieties. These bonds may be covalent, ionic and may involve linking intermediates. The alginates polymers may thus be prepared in predetermined shapes through non-gelling cross-linkers for example.

Modified alginate samples have the formula:

A-X

wherein A is the alginate polysaccharide and X is a modifying moiety. A and X are linked

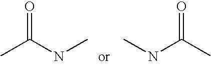

covalently through linkages selected from ester, ether, thioethr, disulfide, amide, imide secondary amines, direct carbon-carbon (C—C) linkages, sulfate esters, sulfonate esters, phosphate esters, urethanes, carbonates, and the like. That is, one or more monomers of an alginate may be covalently linked to a modifying moiety directly or with a spacer. Thus, modified alginate samples may have also the formula:

A-Y—X

wherein A and X have been specified above, Y is a spacer containing alkyl or aryl chains suet as an alkyl group, an alkenyl group, an alkynyl group, an aryl group. In some embodiments, the alkyl group is a C₁-C₁₅, preferably C₁-C₁₀, preferably a C₁-C₅, preferably a C₁-C₃ alkyl, alkenyl alkynyl, or aryl group. A and Y, as well as Y and X, are linked through linkages specified above.

Linkages or linkers may be provided optionally with or without spacers to connect a modifying moiety to a monomer subunit of an alginate polymer. Examples of linkers include, but are not limited to: ester, ether, thioester, disulfide, amide, imide secondary amino, direct carbon-carbon (C—C) linkages, sulfate esters, sulfonate esters, phosphate esters, urethanes, and carbonates, used in combination with or without spacers such as an alkyl group, an alkenyl group, an alkynyl group, an aryl group.

Ester linkages refer to a structure of either:

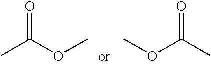

Ether linkages refer to a structure of —O—, thioether linkages refer to a structure of —S—, sidulfide linkages refer to a structure of —S—S—, amide linkages refer to a structure of either

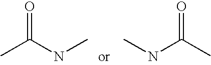

Imide linkages refer to a structure of:

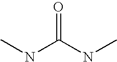

Secondary or tertiary amine linkages refer to:

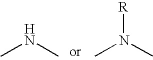

Direct carbon-carbon linkages refer to a structure of —C—C—, sulphonate and sulphate ester linkages refer, respectively, to:

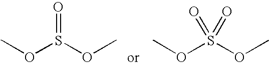

Phosphate ester linkages refer to:

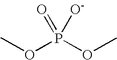

Urethane linkages refer to:

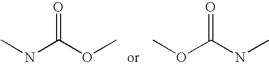

Carbonate linkages refer to:

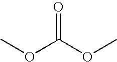

The process of the invention includes one or more steps in which one or more unmodified M residues of alginate are converted to a G residues by enzymatic epimerization reaction Epimerase enzymes are widely known. Examples are derived from Azotobacter vinelandii such as those described in U.S. Pat. No. 5,939,289, which is incorporated herein by reference. Other sources include Pseudomonas syringae (Bjerkan et al J. Biol:chem, Vol. 279, pages 28920-28929, which is incorporated herein by reference) and Laminaria digitata, which are disclosed in international application publication number WO2004065594 published Aug. 5, 2004, which is incorporated herein by reference.

The mannuronan C-5 epimerases, the AlgE enzymes comprises a family of modular proteins encoded by alginate producing bacteria such as Azotobacrer vinelandii. U.S. Pat. No. 5,939,289 discloses the sequences coding these enzymes, a process for preparation of these enzymes and their use to prepare alginates having definite G/M ratio and block structures. These isoenzymes differ in their activity and in the epimerisation pattern they introduce. While AlgE-1 and 6 are effective in generating long G-block, AlgE4introduces only MGM sequences. The former gives strong gel formers while the latter enzyme generates flexible chains (refs). See for example Table 1.

TABLE I
The seven AlgE epimerases from A. vinelandii

| Type | [kDa] | Modulare structure | Products |
|---|---|---|---|
| AlgE1 | 147.2 | 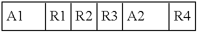 | Bi-functionalG-blocks +MG-blocks |
| AlgE2 | 103.1 | 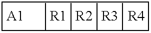 | G-blocks (short) |
| AlgE3 | 191 | 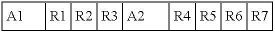 | Bi-functional |
| AlgE4 | 57.7 | 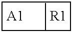 | MG-blocks |
| AlgE5 | 103.7 | 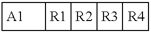 | G-blocks (short) |
| AlgE6 | 90.2 | 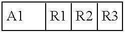 | G-blocks (long) |
| AlgE7 | 90.4 | 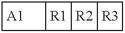 | Lyase activity +G-blocks +MG-blocks |

A-385 amino acids,
R-155 amino acids

All alginates and mannuronans can be epimerized by use of different C-5 epimerases, used singularly or as mixture, in one step or sequentially including varying the order of the chemical and enzymatic steps such as epimerization of the starting alginate prior to substitution followed by additional epimerization. By varying both the degree of substitution and the amount and time of epimerization, different selectively substituted alginate molecules can be obtained. Epimerization reacions can be controlled by controlling temperature, reaction time, the amount of reagents and combinations thereof. For example, in some embodiments, the epimerization reaction is stopped by adding acid, by heating to 90° C. or by adding 50 mM EDTA that seqester the calcium tons necessary for enzyme action. By controlling reactions, the amount of unmodified converted to Gs can be controlled and thus the amount of (G in the final modified alginate can be controlled.

The nature of the starting material also controls the nature of the final product. Using a polymannuronate as a starting material in a process in which modification precedes epimerization provides final products in which only Ms are modified. That is, starting with a polysaccharide containing mannuronic acid residues polymannuronate), it has been discovered that such material can be modified, either on the carboxylic function or on the hydroxyl groups, and subsequently epimerized by use of the C-5 epimerases. Such epimerization occurs on the non-modified residues, leading to an alginate molecule selectively modified on mannuronic acid.

If mannuronan is used as a starting material and modification of residues precedes any enzymatic conversion of Ms to Gs, the modification reaction will lead to mannuronan with substituents randomly distributed along the polymer chain. The amount of modified residues relative to unmodified may be controlled by controlling reaction time, temperature, amounts of reagents and combinations thereof to produce modified mannuronan with the desired degree of modified Ms. In the second step herein, the partially substituted mannuronan is treated with the mannuronan-C-5 epimerases, i.e., the enzymes that converts D-M residues into L-Guluronic acid without breaking the polymer chain. Since the C-5 epimerases are unable to convert substituted M-residues, the end product will be polymers which contain intact G-blocks for calcium binding and junction formation and substituents located exclusively on the M residues which remain in a soluble portion. Here they are free to interact with each other in chemical cross-linking or with exogenous receptors.

In some embodiments, the starting alginate contains both M and G. In this case, the chemical substitution can take place on both M and G residues. Treatment of the partially substituted alginate with enzymes then converts a portion of the unsubstituted M and G residues. An embodiment is an alginate comprising poly MG blocks which is first partially substituted on M and/or U groups and then enzymatically reacted by C-5 epimerization using a G-forming enzyme (i.e. AlgE-1) which has specificity for convening the remaining polyalternating segment of MG.

By controlling the order to steps and reaction rates, the modified alginate polymer produced can have varying degrees of modification, varying levels of modifications of M versus G, varying amounts of unmodified Ms and varying amounts of unmodified Gs. In some embodiments, only Ms are modified. In some embodiments, Ms and Gs are modified.

In some embodiments, less than 10% residues are modified. In some embodiments, less than 20% of residues are modified. In some embodiments, more than 20% of residues are modified. In some embodiments; 10>80% of residues are modified. In some embodiments; 20-60% of residues are modified. In some embodiments, 30-50% of residues are modified. In some embodiments, about 40% of residues are modified.

In some embodiments, less than 260% of residues are unmodified Gs. In some embodiments, more than 20% of residues are unmodified Gs. In some embodiments, 20-80% of residues are unmodified Gs. In some embodiments, 30-60% of residues are unmodified Gs. In some embodiments, 40-50% of residues are unmodified Gs. In some embodiments, about 45% of residues are unmodified Gs.

The modified alginates may be used to prepare alginate gels or fibers by combining the modified alginates a divalent gelling ion such as Ca⁺⁺, Sr⁺⁺, Ba⁺⁺, Zn⁺⁺, Fe⁺⁺, Mn⁺⁺, Cu⁺⁺, Pb, Co, Ni, or combinations thereof.

In some embodiments, the alginate gel is used to encapsulate living cells such as proliferating cells or non-proliferating cells. The cells may be from cell lines or patients/donors. Examples of cells include: pancreatic islets, hepatic cell, neural cells, renal cortex cells, vascular endothelial cells, thyroid and parathyroid cells adrenal cells, thymic cells, ovarian cells, chondrocytes, muscle cells, cardiac cells, stein cells, fibroblasts, keratinocytes or cells derived from established cell lines, such as for example, 293, MDCK and C2C12 cell lines. In some embodiments, encapsulated cells comprise an expression vector that encodes one or more proteins that are expressed when the cells are maintained. In some embodiments, the protein is a cytokine, a growth factor, insulin or an angiogenesis inhibitor such as angiostatin or endostatin, other therapeutic proteins or other therapeutic molecules such as drugs. Proteins with a lower MW, less than about 60-70, are particularly good candidates because of the porosity of the gel-network. In some embodiments, the cells are present as multicellular aggregates or tissue.

In some embodiments alginate fibers are prepared in a process that comprises combining a plurality of modified alginate polymers with a divalent gelling ion and extruding a fiber that comprising cross-linked alginate polymers. In some embodiments, a solid non-crosslinked alginate composition or paste is prepared by forming molding, casting or otherwise shaping a plurality of modified alginate polymers.

In some embodiments, the modification step and/or epimerase step is performed on an already existing alginate get or fiber.

### EXAMPLES

### Example 1

### Preparation of Modified Polymannuronan with 1-Amino-1-Deoxy-Galactose

1-amino-1-deoxy-β-D-galactose (270 mg) was added to a stirred solution of the sodium form of polymannuronan (1.5 g) in 0.2 M 2-[N-morpholino]ethanesulfonic acid (MES) buffer (pH4.5, 400 mL) containing N-hydroxysuccinimide (NHS) (1.3 g) and 1-Ethyl-3-[3-(dimethylamino)-propyl]carbodiimide hydrochloride (EDC) (2.17 g). The solution was stirred for 30 minutes at room temperature. The product was dialyzed against deionized water through a dialysis membrane with a molecular weight cut-off of 12000-14000 for 5 days. The dialyzed product was freeze-dried to obtain the pure galactose derivative of sodium polymannuronan. Yield: 1.45 g. The degree of substitution, calculated from 1H-NMR, was found to be 12% The amide formation by this method was targeted to the carboxylic (uronic) group of the mannuronic acid present in the polymer, Those of skill in the art recognize that the degree of substitution of the product can be varied by use of different ratios of polymannuronan to 1-amino-1-deoxy-galactose in the above-described reaction. The same procedure applies to aminoacids, peptides, different mono- and oligosaccharides, nucleotides and photo-crosslinkable groups bearing an amino group with or without an alkyl or aryl spacer between the molecule and the amino functionality.

### Example 2

### Synthesis of Methacrylate Esters of Polymannuronan

Sodium polymannuronan (3 g) was dissolved in 300 mL of deionized water and cooled to 4° C. in an ice bath. Methacrylic anhydride (23 g) was added dropwise with constant stirring to the cold polymannuronan solution and the pH maintained at 9.0 by addition of suitable quantity of 5M NaOH. The stirring was continued for 24 h at a temperature of 4° C. The reaction product was precipitated in 96% ethanol, centrifuged and washed 3 times with ethanol. The product was then dissolved in water and dialyzed against deionized water through a dialysis membrane with a molecular weight cut-off of 12000-14000 for 3 days. The dialyzed product was freeze-dried to obtain the pure methacrylate derivative of sodium polymannuronan. Yield: 2.6 g. The degree of substitution, calculated from the 1H-NMR is 8%. The ester formation by this method was targeted to the secondary hydroxyl groups present in the monomeric unit. Those of skill in the art recognize that the degree of substitution of the product can be varied by use of different ratios of polymannuronan to anhydride in the above-described reaction. The same procedure applies to suitably modified aminoacids, peptides, different mono- and oligosaccharides, nucleotides and photo-crosslinkable groups

### Example 3

### Epimerization of Modified Polymers by Using AlgE4

The modified polymannuronan sample obtained as described in Examples 1 and 2 (1 g) was dissolved in 50 mM MOPS buffer (ph6.9) containing CaC12 (2.5 mM) and NaCl (10 mM) at a concentration of 2.5 g/L. The G-5 epimerase AlgE4 was then added (enzyme/polymer weight ratio=1/200) and the solution was stirred for 24 h at 37° C. The epimerization reaction was quenched by addition of concentrated HCl to the cold polymer solution to a pH value of 1-2. The mixture was added of NaCl (final concentration 1.5%) and maintained overnight at 4° C. The precipitated product was centrifuged and washed with dilute HCl (0.05M) three times. The product was dissolved in deionized water maintaining the pH slightly above 7. The solution was added of NaCl (final concentration 0.2%) and precipitated with 96% ethanol. The product was filtrated, washed 3 times with ethanol and dialyzed against deionized water through a dialysis membrane with a molecular weight cut-off of 12000-14000 for 3 days. The dialyzed product was freeze-dried to obtain the pure epimerized polymer of modified mannunonan. Yield: 0.85 g. Those of skill in the art recognize that the degree of epimerization can be varied by use of different times of the reaction.

### Example 4

### Epimerization of Modified Polymers by Using AlgE6

The modified polymannuronan sample obtained as described in Examples 1 and 2 (1 g) was dissolved in 50 mM MOPS buffer (H 6.9) containing CaCl₂ (2.5 mM) and NaCl (7 mM) at a concentration of 2.37 g/L. The C-5 epimerase AlgE6 was then added (enzyme/polymer weight ratio 1/20) and the solution was stirred for 48 h at 37° C., The epimerization reaction was quenched by addition of concentrated HCl to the cold polymer solution to a pH value of 1-2. The mixture was added of NaCl (final concentration 1.5%) and maintained overnight at 4° C. The precipitated product was centrifuged and washed with dilute HCl (0.05M) three times. The product was dissolved in deionized water maintaining the pH slightly above 7. The solution was added of NaCl (final concentration 0.2%) and precipitated with 96% ethanol. The product was filtrated, washed 3 times with ethanol and dialyzed against deionized water through a dialysis membrane with a molecular weight cut-off of 12000-1400 for 3 days. The dialyzed product was freeze-dried to obtain the pure epimerized polymer of modified mannuronan. Yield 0.90 g. Those of skill in the art recognize that the degree of epimerization can be varied by use of different times of the reaction,

This procedure gave two types of polymers:

1. Modification of uronic groups with Galactose followed by epimerization d.s.=12%: FG=0.45; FM=0.55; FGG=0.16.

### And

2) Modification of hydroxyl groups with a photocrosslinkable substituent followed by epimerization

Starting material: Polymannuronan modified as reported in Example 2: d.s.=8%, FM=1 Epimerized material: d.s.=8%; FG=0.54; FM=0.46; FGG=0.37.

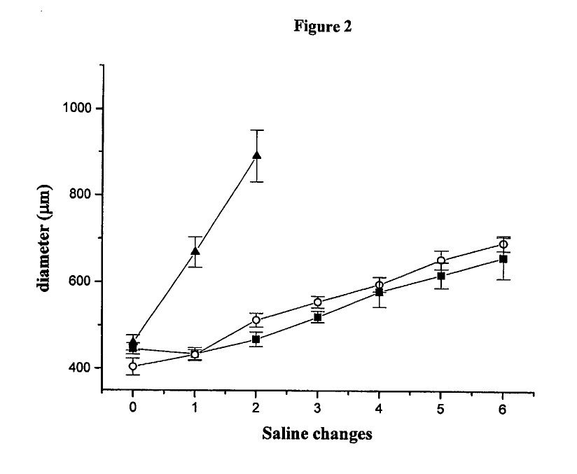

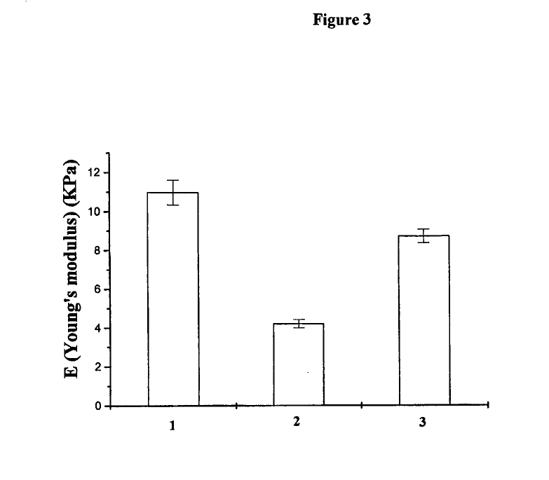

FIGS. 2 and 3 show the effect on the gelling properties of galactosylated and epimerized mannuronan compared to the unmodified and modified (14% of galactose) alginates from Laminaria hyperhorean.

### Example 5

### Epimerization of Chemically Modified Polymers by Using a Combination of AlgE4 and AlgE6

The modified polymannuronan sample obtained as described in Examples 1 and 2 (1 g) was dissolved in 50 mM MOPS buffer (pH 6.9) containing CaCl₂ (2.5 in M) and NaCl (10 mM) at a concentration of 2.5 g/L. The C-5 epimerase AIgE4 was then added (enzyme/polymer weight ratio=1/100) and the solution was stirred for 24 h at 37° C. The C-5 epimerase AlgE6 was then added (enzyme/polymer weight ratio 1/20) and the solution was stirred for 24 h h at 37° C. The epimerization reaction was quenched by addition of concentrated HCl to the cold polymer solution to a pH value of 1-2. The mixture was added of NaCl (final concentration 1.5%) and maintained overnight at 4° C. The precipitated product was centrifuged and washed with dilute HCl (0.05M) three times. The product was dissolved in deionized water maintaining the pH slightly above 7. The solution was added of NaCl (final concentration 0.2%) and precipitated with 96% ethanol. The product was filtrated, washed 3 times with ethanol and dialyzed against deionized water through a dialysis membrane with a molecular weight cut-off of 12000 14000 for 3 days. The dialyzed product was freeze-dried to obtain the pure epimerized polymer of modified mannunonan. Yield: 0.90 g. Those of skill in the art recognize that the degree of epimerization can be varied by use of different times of the reaction to yield polymers with both G-blocks and poly-alternating blocks interspacing the substituted M residues and differs from the AlgE 6 epimerised polymers by lacking MM sequences. This enhances the flexibility of the polymers and leads to higher synresis and lower swelling.

### Example 6

### Preparation of a Polymer Comprising of G-Blocks Interspaced with Substituted PolyMG Sequences

The polymannuronan sample obtained as described in Examples 1 and 2 (1 g) was dissolved in 50 mM MOPS buffer (pH 6.9) containing CaCl₂ (2.5 mM) and NaCl (10 mM) at a concentration of 2.5 g/L. The C-5 epimerase AlgE4 was then added (enzyme/polymer weight ratio=1/100) and the solution was stirred for 24 h at 37° C. The epimerization reaction was quenched by addition of concentrated HCI to the cold polymer solution to a pH value of 1-2. The mixture was added of NaCl (final concentration 1.5%) and maintained overnight at 4° C. The precipitated product was centrifuged and washed with dilute HCl (0.05M) three times. The product was dissolved in deionized water maintaining the pH slightly above 7. The solution was added of NaCl (final concentration 0.2%) and precipitated with 96% ethanol. The product was filtrated, washed 3 times with ethanol and dialyzed against deionized water through a dialysis membrane with a molecular weight cut-off of 12000-14000 for 3 days. The dialyzed product was freeze-dried to obtain the pure epimerized polymer of modified mannuronan. Yield: 0.85 g. Composition Molar fraction of G=0.47 Molar fraction of GG=0 Those of skill in the art recognize that the degree of epimerization can be varied by use of different times of the reaction.

Preparation of Modified PolyMG with 1-Amino-1-Deoxy Galactose

1-Amino-1-Deoxy-β-D-Galactose (270 mg) was added to a stirred solution of the sodium form of modified polymannuronan (1.5 g) in 0.2 M MES buffer (pH4.5, 400 mL) containing NHS (1.3 g) and EDC (2.17 g). The solution was stirred for 30 minutes at room temperature. The product was dialyzed against deionized water through a dialysis membrane with a molecular weight cut-off of 12000-14000 for 5 days. The dialyzed product was freeze-dried to obtain the pure galactose derivative of sodium polymannuronan. Yield: 1.45 g. The degree of substitution, calculated from ¹M-NMR, was found to be 12%. The amide formation by this method was targeted to the carboxylic (uronic) group of the present in the polymer. Those of skill in the art recognize that the degree of substitution of the product can be varied by use of different ratios of poly MG to 1-amino-1-deoxy-galactose in the above-described reaction. The same procedure applies to aminoacids, peptides, different mono- and oligosaccharides, nucleotides and photo crosslinkable groups bearing an amino group with or without an alkyl or aryl spacer between the molecule and the amino functionality.

Epimerization of Modified PolyMG by using AlgE1

The modified polymannuronan sample obtained as described in example 1 and 2 (1 g) was dissolved in 50 mM MOPS buffer (H 6.9) containing CaCl₂ (2.5 mM) and NaCt (75 mM) at a concentration of 2.37 g/L. The C-5 epimerase AlgE1 was then added (enzyme/polymer weight ratio=1/20) and the solution was stirred for 48 h at 37° C. The epimerization reaction was quenched by addition of concentrated HCl to the cold polymer solution to a pH value of 1-2. The mixture was added of NaCl (final concentration 1.5%) and maintained overnight at 4° C. The precipitated product was centrifuged and washed with dilute HCl (0.05M) three times. The product was dissolved in deionized water maintaining the pH slightly above 7. The solution was added of NaCl (final concentration 0.2%) and precipitated with 96% ethanol. The product was filtrated, washed 3 times with ethanol and dialyzed against deionized water through a dialysis membrane with a molecular weight cut-off of 12000-14000 for 3 days. The dialyzed product was freeze-dried to obtain the pure epimerized polymer of modified mannuronan characterized by long 6-blocks interspaced with M or G substituted polyMG sequences. Yield: 0.90 g. Those of skill in the arm recognized that the degree of epimerization can be varied by use of different times of the reaction.

### Example 7

A combination of a chemical and an enzymatic approach has been exploited to obtain an alginate-like polymer bearing β-galactose moieties exclusively on M residues. 1-amino-1-deoxy-galacose was introduced on mannuronan via an amide bond using EDC (1-Ethyl-3-[3-(dimethylamino)-propyl]carbodiimide hydrochloride) and NHS (N-hydroxysuccinimide) as coupling reagents. This polymer has been epimerized by use of two different C-5 epimerases introducing guluronic residues both in alternating and in homopolymeric G (sequences. The grafted alginate selectively modified on M residues has been characterized with ¹H-NMR, HPSEC-RI-MALLS and intrinsic viscosity and its calcium binding ability was detected by means of circular dichroism spectroscopy. The modified material revealed an improvement in mechanical and gel forming and mechanical properties when compared with an alginate sample where the same sugar moiety was introduced on G residues. Finally, the selective modification on M residues resulted in a higher stability of the calcium beads prepared from the grafted alginate.

### Materials and Methods

Commercial sample of sodium alginate isolated from Laminaria hyperborea stipe, LF 10/60, (F_{G}=0.69; F_{GG}=0.56 was provided by FMC Biopolymers (Norway). High molecular weight mannuronan (F_{G}=0.001) was isolated from an epimerase-negative mutant (Alg⁻) of Pseudomonas fluorescens. Purification and deacetylation were carried out as described in Ertesvåg, H,; Skjåk-Bræk, G. in Methods in Biotechnology, 1999, Carbohydrate Biotechnology Protocols; Bucke, C. Ed.; Humana Press Inc., Totowa, N.J., 10, 71, which is incorporated herein by reference. PolyalternatingMG (F_{G}=0.47; F_{GG}=0) was prepared from mannuronan by use of AlGE4 epimerase as described in Hartmann, M.; Duun, A. S.; Markussen, S.; Grasdalen, H.; Valla, S.; Skjåk-Bræk, G. Biochim. Biophys. Acta, 2002, 1570, 104, which is incorporated herein by reference. 1-Ethyl-3-[3-(dimethylamino)propyl]carbodiimide hydrochloride (EDC) and sodium chloride were purchased from Aldrich Chemical Co. (Milwaukee, Wis.). N-hydroxysuccinimide (NHS), 2-[N-morpholino]ethanesulfonic acid (MES) and D-glucono-δ-lactone (GDL) were purchased from Sigma Chemical Co. (St. Louis, Mo.), Calcium carbonate (average particular size 4 μm) was purchased from Merck (Darmstadt, Germany).

### Recombinant Mannuronan C-5 Epimerases

The mannuronan C-5 epimerases were produced by fermentation of these recombinant E. coli strains: AlgE4 in JM 105 and AgE6 in SURE. The enzymes were partially purified by ion exchange chromatography on Q-Sepharose FF (Pharmacia, Uppsala, Sweden) and by hydrophobic-interaction chromatography on phenyl Sepharose FF (Pharmacia). The activity of the enzymes was assayed by measuring the release of tritium to water, when ³H-5-labeled mannuronan was incubated with the enzymes.

### Galactose-Substituted Mannuronan (MGal)

1-amino-1-deoxy-β-D-galactose (galactosylamine) (270 g, 0.2 eq.) was added to a stirred solution of the sodium form of mannuronan (1.5 g) in 0.2 M MES buffer pH4.5, 400 mL) containing NHS and EDC ([EDC]/[Polym]=1.5; [NHS]/[EDC]=1, [Polym] is the molar concentration of glylcopyranoside polymer repeating units). The solution was stirred for 30 minutes at room temperature, the polymer dialyzed (cut-off molecular weight of the membrane approx. 12000) against NaHcO₃ 0.05M for 1 day and then against deionized water until the conductivity was below 2 μS at 4° C. The pH was adjusted to 7, the polymer was filtered through 0.45 μm Millipore filters and freeze-dried yielding a modified mannuronan sample containing 12% of galactose introduced as side-chain group as revealed by ¹H-NMR analysis (degree of substitution (d.s.) calculated from the intensity of the H-1 signal of galactosylamine with respect to the intensity of the anomeric proton of the M residues in the polymer chain) and potentiometric titration.

Epimerization with AlgE4 (MGalE4)

The polymer MGal was dissolved in 50 mM MOPS buffer (pH 6.9) containing CaCl₂ (2.5 mM) and NaCl (10 mM) at a concentration of 2.5 g/L. The C-5 epimerase AlgE4 was added (enzyme/polymer weight ratio =1/100) and the solution stirred for 24 h at 37° C.

Epimerization with AlgE6 (MGalE4E6)

The polymer MGalE4 was dissolved in 50 mM MOPS buffer (pH 6.9) containing CaCl₂ (2.5 mM) and NaCl (75 mM) at a concentration of 2.37 g/L. The C-5 epimerase AlgE6 was added (enzyme/polymer weight ratio 120) and the solution stirred for 48 h at 37° C.,

### Purification of Epimerized Polymers

The epimerization reaction was quenched by addition, to the cold polymer solution, of a 5M NaCl solution (final concentration 1.5%) and of hydrochloric acid (3 M) to an approximate pH value of 1-2. The mixture was stored overnight at 4° C. to aid the precipitation. The precipitate was centrifuged and washed with dilute HCl (0.05M) three times. The precipitate was then dissolved in deionized water maintaining the pH slightly above 7 by addition of dilute sodium hydroxide. The solution was mixed with a 5M solution of NaCl (final concentration 0.2%) and precipitated with ethanol. The precipitate was dissolved, dialyzed (cut-off molecular weight of the membrane approx. 12000) against deionized water until the conductivity was below 2 μS at 4° C., the pH adjusted to 7, filtered though 0.45 μm Millipore filters and freeze-dried.

Galactose-substituted alginate from L. hyperborea (LhypGal)

An alginate sample from L. hyperborea was treated with 1-amino-1-deoxy-galactose as previously reported. A modified alginate bearing 14% of galactose moieties introduced on G residues, as revealed by ¹H-NMR analysis, was obtained.

### ¹H-NMR Spectroscopy

Samples were prepared as described by Grasdalen et al. The ¹H-NMR spectra were recorded in D₂O at 90° C. with Bruker WM 300. The chemical shifts are expressed in p.p.m. downfield from the signal for 3-(trimethylsilyl)propanesulfonate.

### Potentiometry

Potentiometric titrations were performed to determine the equivalent weight of the MGal and MGalE4E6 samples. A Radiometer pHM240 pH-meter equipped with a glass electrode was used. The H⁺ form of the polymers was prepared by dialyzing a 3 g/L solution against 0.1 M HCl overnight. The excess of HCl was removed by exhaustive dialysis against deionized water. The polymer was recovered by freeze-drying. Aqueous solutions of known polymer specific concentration were titrated with 0.1 M NaOH standard solution (Tritisol, Merck). A repeating unit molar mass of 198±4 g/mol and 200±3 g/mol were found for the H⁺ form of MGal and MGalE4E6, respectively, which compared rather well with the theoretical value calculated on the basis of the degree of substitution obtained from NMR (195.3 g/mol).

### Circular Dichroism Spectroscopy

Circular dichroism spectra of the sodium form of the polymers MGal, MGalE4 and MGalE4E6 (see Table 2), respectively, were recorded in deionized water (c˜2*10⁻³ monomol/L) with a Jasco J-700 spectropolarimeter. A quartz cell of 1-cm optical path length was used and the following set-up was maintained: bandwidth, 1 nm; time constant, 2s; scan rate, 20 nm/min. Four spectra corrected for background were averaged for each sample. The spectrum of each sample was recorded prior to and after the addition of a Ca(ClO₄)₂ solution to a ratio [Ca ²⁺]/[Polym]=0.26.

### Bead Formation

Calcium beads from L. hyperborea, LhypGal and MGalE4E-6, respectively, were obtained by letting a 2% (w/V) polymer solution drip into 50 mM CaCl₂ solution. The droplet size was controlled by using a high voltage electrostatic bead generator (7 kV, 10 mL/h steel needle with 0.4 mm-outer diameter, 1.7-cm distance from the needle to the gelling solution). The alginate gel beads obtained were stirred 30 min in the gelling solution prior to use.

### Stability in Saline Solution

The dimensional stability of calcium alginate beads obtained from L. hyperbora, LhypGal and MGalE4E6, respectively) was measured with an inverted light microscope (Zeiss) when ½ mL of gel beads was added to 3 mL saline solution (0.9%). The sample was stirred for 1 i h. The saline solution was replaced several times and the diameter of the capsules was determined (n=25) before each change. Capsules were rinsed with deionized water prior to measurement.

### Gelling Kinetics and Rheological Characterization

Gelling kinetics and dynamic viscoelastic characterization were carried out applying a Stress-Tech general-purpose rheometer (REOLOGICA instruments AB, 22363 Lund, Sweden). Briefly, to a 1.5% solution of L. hyperborea, LhypGal and MGalE4E6 (see Table 2), respectively, CaCO₃ (20 mM) and GDL (40 mM) were added and the mixture was stirred for 30 sec prior to the measurements. These experiments were performed with a serrated plate-plate (d=40 mm) measuring geometry with T=20° C. and gap=1.00 mm. The kinetics of gelation was determined by repeated determination of G′ and G″ (ω=6.28 rad*s⁻¹) at intervals of 3 minutes for approximately 18 h. The dynamic viscoelastic characterization was carried out 24 h after inducing gelation by determining the frequency dependence of the storage (G′) and loss moduli (G″). Frequency sweeps were performed at a constant strain (0.001) in the frequency range 0.01 to 50 Hz. The samples were sealed with a low-density silicon oil to avoid adverse effects associated with evaporation of the solvent throughout the gelation experiments.

Preparation Of gels Cylinders and Syneresis

Homogeneous calcium gels from L. hyperborea, LhypGal and MGalE-4E6, respectively, were prepared by blending the polymer solution with an inactivated form of Ca²⁺ (CaCO₃) followed by the addition of the slowly hydrolyzing D-glucono-δ-lactone (GDL), maintaining a molar ratio GDL/Ca²⁺=2. The final concentration of polymer was 1% (w/V) in all cases.

Syneresis of the Ca-alginate get was determined as the weight reduction of the gels with respect to the initial weight, calculated assuming a density value of 1. Aliquots of Ca-polymer gelling solutions, prepared as described above, were cured in 24 wells tissue culture plates having a diameter of 16 mm and height of 18 mm (costar, Cambridge, Mass.). The gels were taken out from the wells after 24 h and their weight measured. The syneresis was calculated as (1−W/W₀)*100, were W and W₀ are the final and initial weight of the gel cylinders, respectively.

The Young's modulus (E) of the resulting gels was calculated from the initial slope of the force/deformation curve as measured with a Stable Micro Systems TA-XT2 Texture analyzer at 20° C. For all gels exhibiting syneresis, the final polymer concentration was determined and E was corrected adapting E∝c².

### Viscosity Measurements

Reduced capillary viscosity of the sodium form of samples MGal, MGalE4 and MGalE4E6 (see Table 2), respectively, was measured in 0.1M NaCl at 25° C. by using a Schott-Geräte AVS/G automatic apparatus and an Ubbelohde type viscometer. Intrinsic viscosity values were determined by analyzing the concentration dependence of the reduced specific viscosity (ηₛₚ/c) and the reduced logarithm of the relative viscosity (lnηᵣₑₗ/c) by using the Huggins (equation 1) and Kraemer (equation 2) equations, respectively.

ηₛₚ/c=[η]+k′[η]²c eq. 1

(lnηᵣₑₗ)/c=[η]−k″[η]²c eq. 2

where k′ and k″ are the Huggins and Kraemer constants.

High Performance Size-Exclusion Chromatography Combined with Multiple Angle Laser Light Scattering (HPSEC-RI-Malls)

The HPSEC-RI-MALLS system consisted of an online degasser (Shimadzu DGUA-4A), a pump (ShimadzuLC-10AD) and 3 serially connected columns (TSK GEL G6000/5000/4000 PWXL). The eluent was (0.05M Na₂SO₄ with 0.01M EDTA pH6) at 0.5 mLh/min. Detectors were refractive index (RI), UV monitor (Pharmacia LKB UV-M II, Amersham Pharmacia Biotech. Uppsala, Sweden) and multiple angle laser light scattering (MALLS-Dawn DSP equipped with a He—Ne laser 632.8 nm, Wyatt Technology Corp., Santa Barbara, Calif., USA). Samples were dissolved at a concentration of ≈1 mg/mL in 0.05M Na₂SO₄ with 0.01M EDTA at pH=6 and filtered through 0.22 μm filters before injection of 100 μL. Data for molecular weight determination were analyzed using ASTRA software (Version 4.70.07, Wyatt Technology Corps, Santa Barbara, Calif., USA) The refractive index increment (dn/dc) used was 0.15.³¹ The angular fit was based on the Debye procedure, weight-average molecular weight M_{w} and number-average molecular weight Mₙ, were obtained following a 1^{st} order polynomial curve fitting of logM (M=molecular weight) versus elution volume.

### Results and Discussion

### Synthesis and Characterization

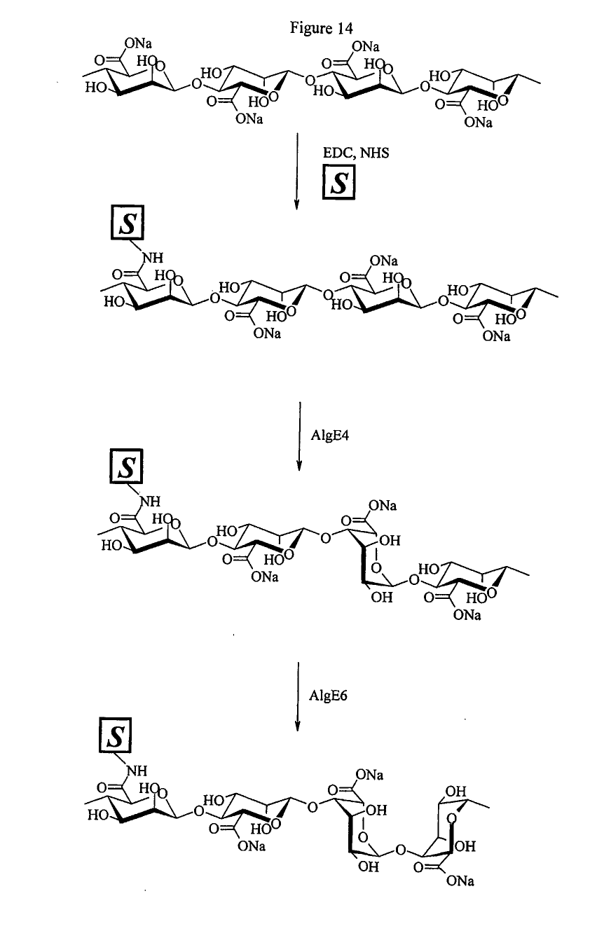

It has been previously reported that the introduction of 1-amino-1-deoxy-β-galactose on alginate chain affects primarily the G residues, influencing both the gelling ability and stability as well as the conformation of the polymer chain. An appealing improvement would be represented by the possibility of a selective introduction of side-chain groups on mannuronic (M) residues. Considering that these groups are not involved in the get formation, the calcium-binding and gelling properties of such selectively modified alginate would be unaffected. However, given the similarity of the uronic functionalities, to the best of the authors' knowledge no strategy based on protecting groups is suitable for this purpose. In order to overcome this problem, a sequential chemical modification of mannuronan followed by two epimerizations induced by C-5 epimerases have been considered (FIG. 14).

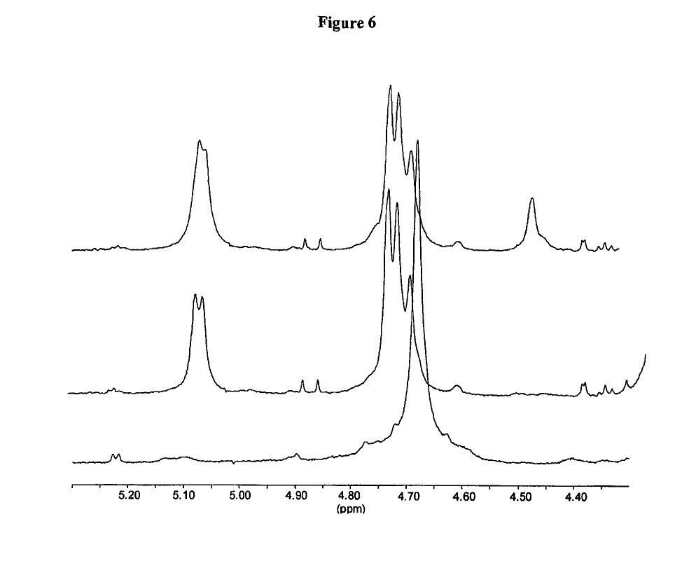

In the first step, 1-amino-1-deoxy-galactose (galactosylamine) was introduced, via an N-glycosidic bond, on the uronic groups of M residues in mannuronan. The coupling reaction between alginate and galactosylamine was performed exploiting the condensing agent EDC in presence of NHS, that already proved to be successful. The ¹H-NMR spectrum of the galactose-substituted mannuronan, MGal, is reported in FIG. 6. As previously noted, upon introduction of galactosylamine moieties on M residues, a newly formed peak is detectable at around 4.75 ppm, arising from the anomeric proton of the sugar present as side chain. The degree of substitution obtained from the area of this peak (12%) is in good agreement with the value obtained from the potentiometric titration of the H⁺ form of the polymer (14%).

Starting from the galactose-substituted mannuronan, guluronic residues have been introduced in the polymeric chain by two successive epimerization reactions performed by use of the enzymes AlgE4 and AlgE6. At variance with previous work, the two enzymes were used separately, in view of the different sodium chloride concentration required to achieve the highest epimerization efficiency. In the first epimerization reaction, the sample MGal has been treated with AlgE4 for 24 h and a residues have thereto been introduced in tong alternating MGM sequences (FIG. 14), as expected provided the mode of action of the epimerase. In fact, in the ¹H-NMR spectrum of the epimerized material, i.e. MGalE4 (FIG. 6), the presence of the peak at ˜5.07 ppm, arising from the anomeric proton of the newly introduced G residues, is clearly detectable. The overall content of G residues (F_{G}), evaluated from the area of the latter peak, was found to be 0.33 (Table 2). Furthermore, it is important to notice the increase in complexity of the spectrum in the region spanning from 4.8 to 4.65 ppm induced by the presence of the H-5 signals belonging to the G residues in alternating sequences.

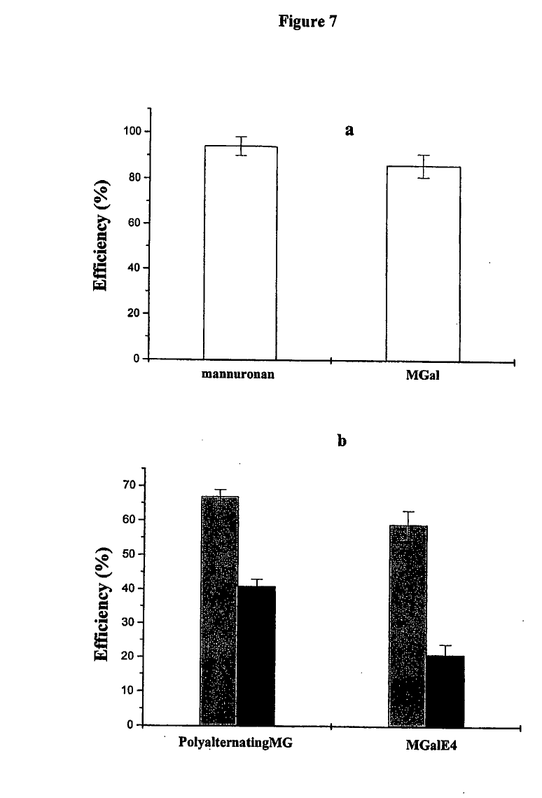

Due to the possibility to discriminate between galactosylamine linked on 1 residues in homopolymeric or in alternating sequences, a hindrance of the epimerization reaction on the modified M residue and on the neighboring group was disclosed, as easily predictable. In fact, no signal located at ˜4.9 ppm, belonging to the anomeric proton of galactosylamine introduced on an M neighboring a C residue, was detected, proving that the M residue neighboring a modified M moiety is not available for epimerization. Based on this consideration, the overall epimerization achieved in the case of MGalE4 was compared with the result obtained for an AlgE-4-treated mannuronan. FIG. 7a reports the efficiency (%) of the enzyme expressed as ratio between the experimental and the theoretical C residues content, the latter calculated assuming a full epimerization of all available M residues to produce strictly alternating sequences. In the case of AlgE4-treated mannuronan, the final G content was found to be 0.47 in strictly alternating sequences. Considering a theoretical maximum value of 0.50 for this substrate, an enzyme efficiency of 94% was calculated. In the case of sample MGalE4, the galactose-modified residues and the neighboring M groups are not available for the epimerization reaction: this fact leads to a theoretical maximum amount of G residues introduced (F) equal to 0.38. It can be therefore concluded that, as the enzyme activity is reduced to 86% in the latter case, the presence of galactose residues as side-chains brings about only a small effect on the epimerization reaction.

The second epimerization, that yields sample MGalE4E6, was performed using epimerase AlgE6 in order to introduce homopolymeric G sequences (FIG. 14). FIG. 6 reports the anomeric region of the ¹H-NMR spectrum of the sample N4GalE4E6 The newly formed signal at ˜4.45 ppm, arising from the H-5 proton of a C residue in homopolymeric sequences, proves the presence of both alternating and homopolymeric G sequences in sample MGalE4E6 which bears 12% of galactose moieties exclusively on M residues. The content of monads and diads of sample MCalE4E6 is reported in Table 2. It is important to underline the presence of as much as 16% of CG diads, an essential feature for the formation of calcium gels.

Some of the signals of the polymer chain in sample MGalE4E6 are overlapped with the signal of the galactose moiety present as a side-chain; this prompts to check the degree of substitution by an independent method. Thus, a potentiometric titration on the H⁺ form of this polymer was performed. The degree of substitution calculated in the latter way (15%) confirmed that no degradation of the N-glycosidic bond took place during neither the epimerization nor the purification of the final product.

In order to evaluate the efficiency of the epimerase AlgE6 on the sample MGalE4, a strictly alternating MG polymer was treated in the same reaction conditions (FIG. 7b). Under the same assumption reported above, one should conclude that the presence of the galaclosylamine in the polymeric chain does not dramatically hamper the introduction of additional G residues in the polymer. In fact, as reported in FIG. 7b, a slight decrease (59%) was experienced for the efficiency of AlgE6 on the galactose-modified polymer when compared to that observed for polyalternatingMG sample (67%). However, the effect of the side-chain is more pronounced on the introduction of GG diads, with an efficiency of the enzyme equal to 21% on MGalE4, compared to 41% displayed by the same enzyme on the polyalternatingMG sample.

Table 2 summarizes the composition of the three samples, in terms of both monads and diads. Being both alternating and homopolymeric C sequences present, sample MGalE4E6 can be described as an alginate-like molecule bearing 12% of galactosylamine moieties selectively on M residues.

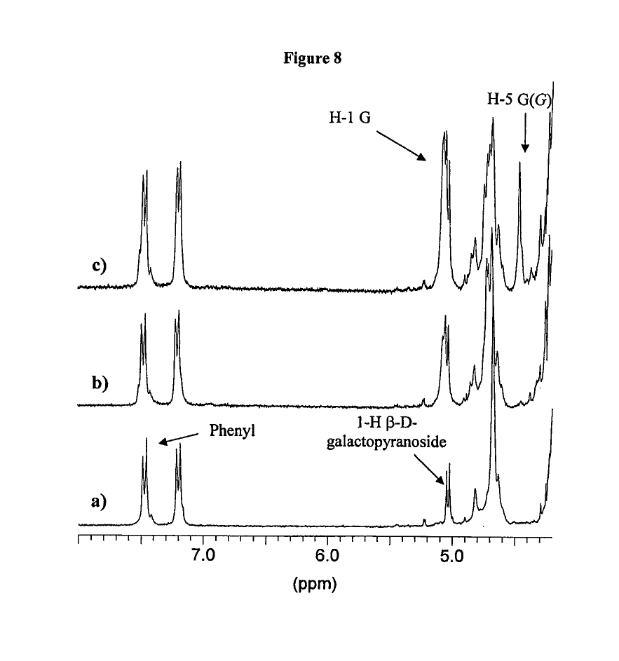

In order to preliminary explore the effect on the epimerization of a spacer introduced between the polymer and the sugar moiety, p-AminoPhenyl-β-D-galactopyranoside (pNH₂PhβGal) was linked on mannuronan polymer chain. This modified polymer, achieved by means of the EDC/NHS chemistry (see Materials and Methods) using 0.3 equivalents of pNH₂PhβGal, was epimerized under the same reaction conditions reported for MGal and the resulting samples were analyzed by ¹H-NMR. From the spectra reported in FIG. 8(a-c) it can be noted that despite the relatively high degree of substitution (d.s.=18%), a notable epimerization has been achieved. In fact, a quantitative analysis of the ¹H-NMR spectra reported in FIG. 8 revealed, concerning the introduction of G residues on the pNH₂PhβGal-modified substrate, a efficiency of 82% and 56% in the case of AlgE4 and AlgE6, respectively, thus proving that the rigid spacer group, represented by the phenyl moiety, does not significantly affect the C-5 inversion of unmodified M residues. In addition, it should be noted that the treatment with AlgE4 and AIgE6 does not cleave the amide bond between the polymer and the side chain group, as proved by the presence of the easily detectable resonances belonging to the aromatic ring in the ¹H-NMR spectrum of the epimerized samples (see FIGS. 8b and c).

A preliminary evaluation of the molecular details of samples MGal, MGalE4 and MGalE4E6 was obtained by means of intrinsic viscosity and SEC-MALLS measurements (Table 2). It is to be stressed that both techniques revealed a decrease of the molar mass as a consequence of the epimerization, likely stemming from a slight lyase activity of the enzymes. In spite of this degradation, the polymer produced from the present chemo-enzymatic approach, i.e. MGalE4E-6, presents a relatively high molecular weight (183000).

An evaluation of the persistence length, q,³⁶ of the samples MGal, MGalE4 and MGalE4E6 was attempted using equation 3, derived from the equivalent model (“nondraining” theory) by Flory and Fox.³⁷ Assuming alginate as a relatively stiff molecule (wormlike chain), the persistence length can be estimated from the value of the intrinsic viscosity and the molecular mass:

$$q = \frac{1}{2} * [ ( \frac{1}{D P * l} ) * {( \frac{[ η ] {M}_{w}}{Φ} )}^{\frac{2}{3}} ] \qquad eq . 3$$

where DP represents the degree of polymerization, 1 is the virtual bond length, and Φ is a function of the spatial distribution of the chain molecule units.³⁸ Equation [3] is strictly valid for monodisperse systems; moreover, it assumes that Φ is a universal constant, or, at least, that it is constant in a given group of different polymers under consideration. Under these hypotheses,²¹ very similar values of q were obtained (12.2±1.2 nm, 13.10.6 nm and 14.4±0.3 nm for MGal, MCalE4 and MGalE4E6, respectively) suggesting, at a qualitative level, that the epimerization of M residues does not significantly alter the stiffness of these galactose-modified polymers.

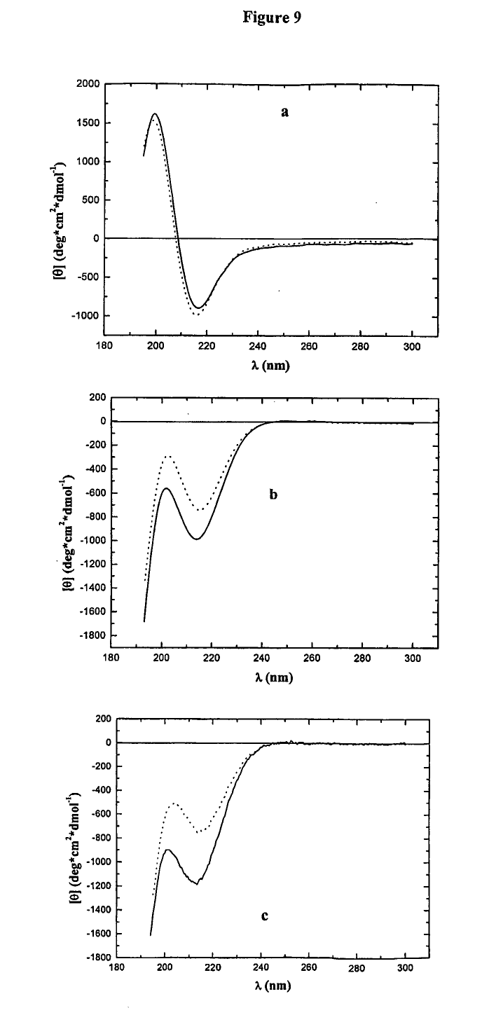

The chirooptical properties of samples MGal, MGalE4 and MGalE4E6, respectively, were investigated by circular dichroism (FIG. 9 a-c). It can be noted that a different profile of the molar ellipticity as a function of wavelength is disclosed by the three samples, stemming from the introduction of G residues in the polymeric chain. In fact, it is well known that the two sugar components of alginate display different chirooptical behavior, the overall CD spectrum of the polymer being dependent upon the relative amount and sequence pattern of M and G moieties. In particular, CD spectra of GG, MM and MG sequences display differences in position, sign and intensity of the peaks. Circular dichroism can also provide a useful, although qualitative, information regarding the binding of divalent cations, such as calcium, by the three polymers above reported, i.e. MGal, MGalE4 and MGalE4E6. The strong coordination of the divalent cation by the uronic moieties of the polymer brings about a change in conformation of the Ca-binding sequences. The latter leads to a modification of the electronic environment of the carboxylate groups, detected as a variation of the overall CD spectrum of the polymer sample. The CD spectra of MGal, MGalE4 and MGalE4E6, respectively, were recorded prior and after the addition of a know and equal amount of calcium and the results are reported in FIG. 9 (a-c). In particular, it can be noted that sample MGal did not display relevant changes in the spectrum upon addition of calcium (FIG. 9a), therefore excluding the possibility of a specific coordination of the calcium ions by homopolymeric M sequences. In contrast, by treating the sample MGalE4E6 with an equivalent amount of calcium, a notable change in the spectrum was detected (FIG. 9c) explained by the formation of conformationally ordered homopolymeric (3 sequences, the so-called “egg-box” structures, that only occur in MGalE4E6. This result proves the ability of such selectively modified and epimerized material to cooperatively bind calcium. It is noteworthy that, as reported in FIG. 9b, also the polymer MGalE4 shows appreciable changes upon addition of calcium, despite the complete lack of GG diads. Although further analyses are required, the formation of interchain junctions between long regular alternating sequences induced by the presence of calcium could be proposed to account for the observed behavior, as already suggested by Morris and co-workers.

### Gel Formation and Properties

In order to propose the selectively modified alginate MGalE4E6 as a suitable bioactive biomaterial, the physical properties of its calcium-gels, i.e. gelling kinetics, viscoelastic behavior and Young' modulus, have been measured. In particular, calcium-hydrogels from sample MGalE4E6 were compared with those obtained fin sample LhypGal, synthesized as previously reported. It is important to underline that while the former bears 12% of 1-amino-1-deoxy-galactose exclusively on M residues, the latter is characterized by the presence of a similar content (14%) of the same residues located on C moieties. Unmodified alginate from L. hyperborea was used in this comparison as a standard gel-forming material.

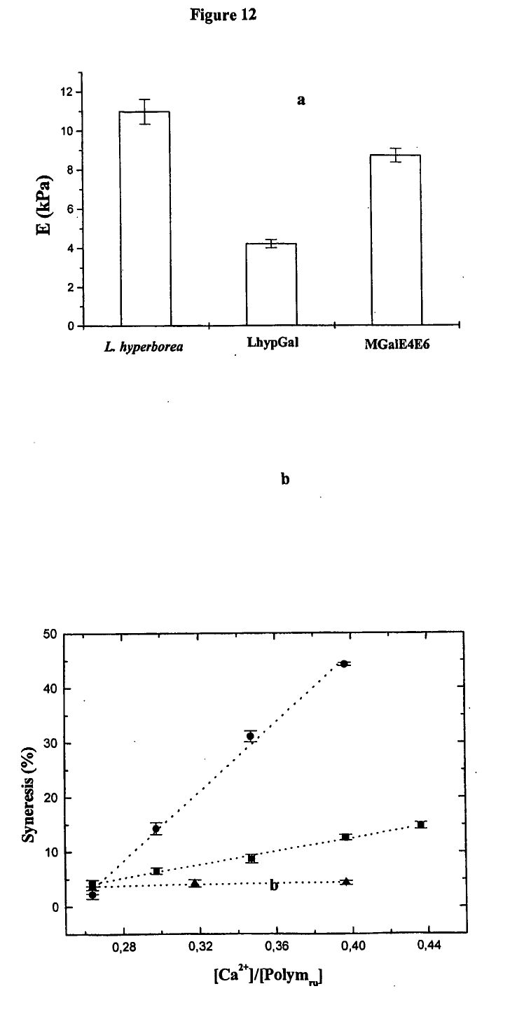

The gel forming kinetics for samples MGalE4E6, LhypGal and alginate form L. hyperborea, respectively, was studied by addition to the polymer solution of calcium ions in an inactivated form (CaCO₃) followed by the slow-hydrolyzing lactone GDL. The ratio between the moles of calcium added and the moles of polymer repeating units was equal to 0.26 for all the three samples, in order to limit the syneresis of the gels (see FIG. 12b).

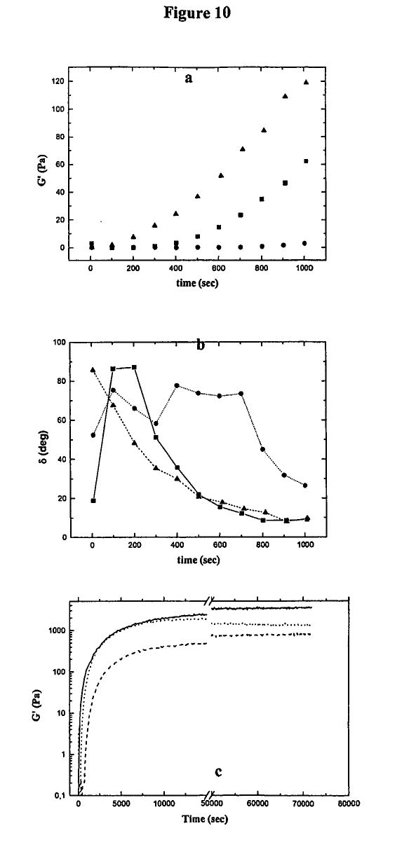

In this “internal gelation” process, the (slow) hydrolysis of GDL releases protons that convert the insoluble CaCO₃ in HCO₃⁻ thus providing the free calcium ions required for the gel formation. The delay between the mixing of the lactone and the gel formation allows the investigation of the formation and curing of the hydrogel in the rheometer (FIG. 10a-c).

FIG. 10a reports the variation of the storage modulus (G′) of L. hyperborea, LhypGal and MGalE6, respectively, in the first 1000 seconds of the gel-forming process. The data show that the introduction of galactose moieties on G residues in alginate strongly affects the kinetics of the gel formation. In fact, from the comparison between LhypGal and the unmodified alginate sample from L. hyperborea, it can be stressed that while the former does not show a significant variation of the G′ value in the first 1000 seconds, the latter discloses a 16-fold increase of the storage modulus. Conversely, the sample MGalE4E6, bearing an amount of galactose similar to that of LhypGal but introduced selectively on M residues, displayed a remarkable increase of the storage modulus during the same observation time, showing a faster gel formation when compared to galactose-modified alginate from L. hyperborea. The remarkable increase of the storage modulus in the case of sample MGalE4E6 could be traced back to the high amount in the polymer of long alternating sequences, which likely lead to a faster and more efficient formation of the junctions.

These considerations are confirmed by FIG. 10b, where the variation of the phase angle (6) recorded during the first 1000 seconds of the gel formation is reported for L. hyperborea, LhypGal and MGalE4E6, respectively. Once more, the introduction of side-chains on the G residues impairs the gel formation of LhypGal. On the contrary, by exploiting the chemoenzymatic approach and achieving a selective substitution on the non-gel forming M residues, i.e. for sample MGalE4E6, the gel forming properties of the polymer are unaffected.

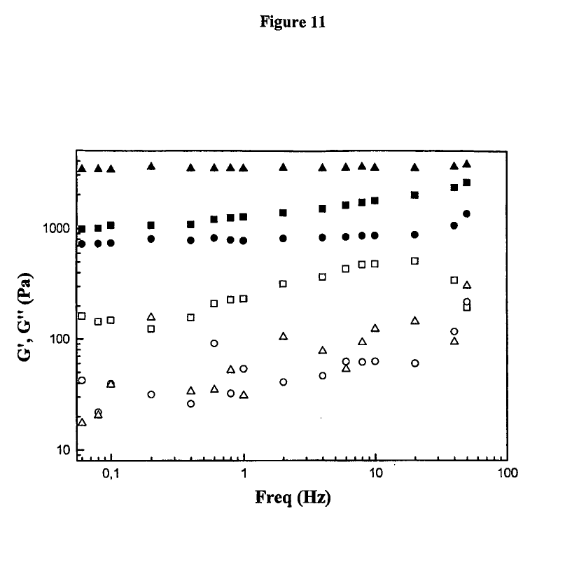

The curing of the gel obtained by internal gelation was followed for approximately 7×10⁴ seconds for L. hyperborea, LhypGal and MGalE4E6, respectively, obtaining stable gels in all the three cases, as shown in FIG. 10c. After the complete formation of the gel, mechanical spectra were measured for L. hyperborea, LhypGal and MGalE4E6 samples, respectively (FIG. 11). In all the three cases, the storage modulus (G′) is always higher than the loss modulus (G″) over the entire range of w, fulfilling the very first requirement in order to define such materials as gels. It is noteworthy that, in the case of sample MGalE4E6, the independence of G′ from the frequency, coupled with the approximately 100-fold difference between G′ and G″, describes this system as a strong gel

In order to obtain a further evaluation of the differences in the physical properties of the hydrogels from the three alginate samples, the Young's modulus for gel-cylinders obtained from MGalE4E6, LhypGal and unmodified L. hyperborea alginate sample, respectively, was measured (FIG. 12a), For a quantitative comparison of the three samples, a constant ratio of 0.59 between moles of Ca²⁺, ions and moles of G residues available for calcium chelation was used. Thus, gel cylinders from MGalE4E6, LhypGal and alginate from L. hyperborea were prepared using different concentrations of calcium carbonate for each polymer, i.e. 13.3, 16 and 22 mM respectively.

It is important to notice that, starting from the value of the unmodified alginate sample (˜1 kPa), the introduction of the galactosylamine moieties on the G residues dramatically affects the gel strength, with a decrease to 4.2 kPa of the Young's modulus for LhypGal however, the introduction of the side-chain galactose on mannuronan followed by two epimerization reactions products better results, in terms of gel strength. In fact, a Young's modulus of 8.7 kPa was measured for sample MGalE4E6 stressing on the importance of the selective modification of polymeric chain.

Sample MGalE4E6 displayed also a remarkable syneresis induced by the amount of calcium (CaCO₃) added, as reported in FIG. 12b. The syneresis of a gel is a phenomenon that macroscopically is characterized by a slow, time-dependent, shrinking, resulting in a partial exudation of liquid. Syneresis has been proposed to be generated by lateral associations of polymeric chains after gel formation and it has already been related to the amount of alternating sequences present in the alginate sample. In FIG. 12b, the syneresis (%) against the ratio calcium/polymer repeating units was plotted for samples MGalE4E6, LhypGal and L. hyperborea, respectively. It can be noted that the epimerized material, i.e. MGalE4E6, shows a higher dependence of the syneresis on the amount of CaCO₃ dispersed in the solution as compared to the unmodified sample from L. hyperborea. This behavior can be explained by taking into account the higher amount of alternating MGM sequences present in the former polymer. In contrast, the G-modified alginate sample from L. hyperborea source, i.e. LhypGal, does not show any dependence of the syneresis on the calcium concentration: in the latter situation the presence of bulky galactose moieties on G residues sterically hinders the lateral association of the polymeric chains in the gel, thus preventing the de-swelling effect.

### Capsule Formation and Stability

Particular attention has been addressed to the ability of sample MGalE4E6 to form capsules. It was noted that on letting a 2% aqueous solution of MGalE4E6 to drip into 50 mM calcium chloride solution, stable capsules were obtained. The diameter of such capsules, controlled by use of an electrostatic bead generator (see Materials and Methods section), was found to be 404±19 μm (n=20).

The stability of the capsules obtained from sample MGalE4E6 was tested by measuring the variation of the dimension (diameter) upon treatment with saline solution (NaCl 0.9 %). For comparison, the stability of capsules obtained from unmodified L. hyperborea and from sample LhypGal was considered.

The capsule is an ionic gel, the volume of which is governed mainly by a positive osmotic pressure (swelling) which is counterbalanced at equilibrium by a negative pressure due to elasticity of the network, the latter being related to the number of cross-links in the gel.

By treating the capsules with an excess of Na⁺ counterions, i.e. a saline solution, a competition between monovalent and divalent cations takes place eventually leading to a displacement of the calcium ions in the capsule. The overall effect of such treatment is a decrease of the number and length of the G junctions accounting for an increase in diameter of the capsules. Therefore, the higher the dimensional variation for a given number of saline shifts, the lower the stability of the capsule.

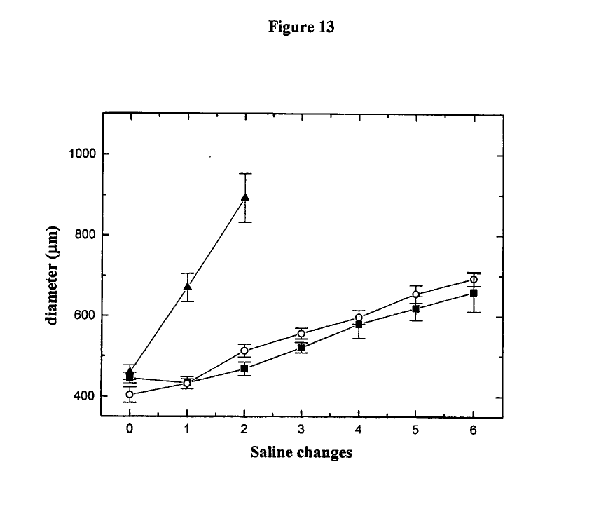

FIG. 13 reports the effect of a repeated replacement of the saline solution on capsules obtained from L. hyperborea alginate, LhypGal and MGalE4E-6, respectively. From the comparison between the unmodified L. hyperborea and the sample bearing 14% of galactose introduced on G residues, i.e. LhypGal, it is to be stressed that in the latter case a net decrease of stability is experienced, as already discussed. If fact after 2 saline solution changes, capsules from sample LhypGal displayed a 2-fold increase in diameter while capsules obtained from unmodified alginate from L. hyperborea showed just a 1.1-fold increase. This effect can be traced back to the presence of side-chain moieties on the guluronic residues in alginate, leading to a substantial impairment of its calcium binding properties.

On the contrary, capsules from MGalE4E6 displayed a remarkable stability, with a 1.3-fold increase in diameter after two saline changes. The higher stability shown by this sample compared to the G-modified material LhypGal, can be explained considering that in the former polymer) the introduction of the side-chain groups affect exclusively the M residues. Such selective modification on residues not involved in the gel formation does not hamper the binding of calcium by the alginate sample, leading to more stable capsules. In addition, a role of long alternating sequences in the stabilization of the capsules can also be proposed, as already reported.

### CONCLUSIONS

The availability of structurally pure mannuronan and of different C-5 epimerases allowed the devising a new strategy for producing alginate-like molecules selectively modified on M residues. The chemo enzymatic approach was tested on the production of a new bioactive biomaterial which bears galactose residues exclusively on mannuronic moieties. The effect of the epimerases on the galactose-modified material was analyzed by ¹H-NMR and the resulting polymers were analyzed by means of intrinsic viscosity, SEC-MALLS and circular dichroism spectroscopy.

Rheological measurement on the modified and epimerized material pointed out on the benefit to the mechanical properties of a selective introduction of side-chain groups on M residues, in particular in comparison with an alginate sample similarly modified on G residues.

By presenting galactose moieties, the modified and epimerized material can be proposed as a new bioactive biomaterial for the encapsulation of hepatocytes where the mechanical and swelling properties of the alginate gels are improved with respect to the modified alginate from L. hyp. sample. It is however important to notice that such chemo enzymatic approach presents a wide applicability, rendering it particularly appealing and opening new opportunities towards the production of novel biomaterials. In conclusion, the modification of mannuronan followed by epimerization can be proposed as a reliable and new methodology in order to obtain selectively modified materials with tailor-made structural and physical properties.

TABLE 2
Composition, in terms of monadic and diadic content, intrinsic viscosity and molecular
weight of the polymers MGal, MGalE4 and MGalE4E6

| | | | | | | | | | | d |
|---|---|---|---|---|---|---|---|---|---|---|
| | | | | | | [η] | | | M_{W} | (M_{W}/ |
| 1. Sample | F_{G} | F_{M} | F_{GG} | F_{GM/MG} | F_{MM} | (dL/g)^{a} | k′ | k″ | (g/mol.)^{b} | M_{N})^{c} |
| MGal | 0 | 1 | 0 | 0 | 1 | 11.98 | 0.424 | 0.12 | 448000 | 1.5₄ |
| MGalE4 | 0.33 | 0.67 | 0 | 0.33 | 0.34 | 9.34 | 0.393 | 0.130 | 236200 | 1.6₈ |
| MGalE4E6 | 0.45 | 0.55 | 0.16 | 0.29 | 0.26 | 8.85 | 0.372 | 0.141 | 183200 | 1.7₃ |

F_{G} denotes the proportion of alginate consisting of guluronic acid.
F_{GG} indicates the proportion of alginate consisting of guluronic acid in blocks of dimers, whereas
F_{MM} indicates the proportion of alginate consisting of mannuronic diads.
F_{GM/MG} indicates the proportion of alginate consisting of mixed sequences of guluronic and mannuronic acid.
^{a}Solvent: NaCl 0.1M, T = 20° C., k′ and k″ represent the Huggins and Kraemer constants, respectively.
^{b}Weight average molecular weight and
^{c}polydispersity index as measured by HPSEC-RI-MALLS.

1. A process for preparing a modified alginate polymer comprising the steps of:
a) covalently attaching a modifying moiety to one or more unmodified monomeric subunits of an alginate polymer with or without a linker; and
b) changing one or more unmodified mannuronic (M) monomeric subunits of the alginate polymer to one or more unmodified guluronic (G) monomeric subunits by an enzymatic epimerization reaction.

2. The process of claim 1 wherein the modifying moiety is selected from the group consisting of: a monosaccharide, an oligosaccharide, a mononucleotide, an oligonucleotide, an amino acid, a peptide and a protein.

3. The process of claim 1 wherein the modifying moiety is selected from the group consisting of: galactose and oligomers thereof, mannose and oligomers thereof, sLe^{x} (NeuAcα2-3Galβ1-[4Fucα1-3]GlcNAc), GlcNAc, HA-oligomers (hyaladhesins; hyaluronan binding proteins), RDG peptides, YIGSR peptides, REDV peptides, IKVAV peptides, KHIIFSDDSSE peptides, and KRSR peptides.

4. The process of claim 1 wherein the enzymatic epimerization reaction uses an epimerase enzyme derived from Azotobacrer vinelandii; Pseudomonas syringae or Laminaria digitara.

5. The process of claim 1 wherein the enzymatic epimerization reaction uses an epimerase enzyme selected from the group consisting of: Azotobacter vinelandii AlgE1, Azotobacter vinelandii AlgE2, Azotobacter vinelandii ALgE3, Azorobacter vinelandii AlgE4, Azotobacter vinelandii AlgE5, Azotobacter vinelandii AlgE6, and Azotobacrer vinelandii AlgE7.

6. The process of claim 1 wherein step a) is performed prior to step b).

7. The process of claim 1 wherein all unmodified monomeric subunits of the alginate polymer are unmodified M monomeric subunits prior to step a).

8. The process of claim 1 wherein the modified alginate polymer comprises 40-50% unmodified G monomeric subunits following steps a) and b).

9. A process of preparing an alginate gel or fiber, the process comprising combining, in a solvent, a plurality of alginate polymers of claim 1 with a divalent gelling ion.

10. The process of claim 9 wherein the divalent gelling ion is calcium, strontium or barium.

11. A process of preparing an alginate gel according to claim 9, wherein said alginate gel further comprises one or more living cells.

12. The process of claim 1 wherein said alginate get further comprises one or more living cells selected from the group consisting of: pancreatic islets, hepatic cells, neural cells, renal cortex cells, vascular endothelial cells, thyroid and parathyroid cells, adrenal cells, thymic cells, ovarian cells, chondrocytes, muscle cells, cardiac cells, stem cells, fibroblasts, keratinocytes or cells derived from established cell lines, sick as for example, 293, MDCK and C2C12 cell lines.

13. A modified alginate polymer comprising unmodified mannuronic (M) monomeric subunits and unmodified guluronic (G) monomeric subunits; wherein only M monomeric subunits are modified and wherein modifications comprise a modifying moiety other than an acetyl group attached to one or more mannuronic (M) monomeric subunits of the alginate polymer with or without a linker.

14. The modified alginate polymer of claim 1 wherein the modifying moiety is selected from the group consisting of, a monosaccharide, an oligosaccharide, a mononucleotide, an oligonucleotide, an amino acid, a peptide and a protein.

15. The modified alginate polymer of claim 1 wherein the modifying moiety is selected from the group consisting of: galactose and oligomers thereof, mannose and oligomers thereof, sLe^{x} (NeuAca2-3Galβ1-[4Fucα1-3]GlcNAc), GlceNAc, HA-oligomers (hyaladhesins; hyaluronan binding proteins), RDG peptides, YIGSR peptides, REDV peptides, IKVAV peptides, KHIFSDDSSE peptides, and KRSR peptides.

16. The modified alginate polymer of claim 1 wherein the modified alginate polymer comprises 40-50% unmodified G monomeric subunits.

17. An alginate gel or fiber comprising a plurality of alginate polymers of claim 1 cross-linked by divalent gelling ions.

18. The alginate gel or fiber of claim 6 wherein the divalent gelling ion is calcium, strontium or barium.

19. An alginate gel of claim 6 wherein said alginate gel further comprises one or more living cells.

20. The alginate gel of claim 8 wherein said alginate gel further comprises one or more living cells selected from the group consisting of: pancreatic islets, hepatic cells, neural cells, renal cortex cells, vascular endothelial cells, thyroid and parathyroid cells, adrenal cells, thymic cells, ovarian cells, chondrocytes, muscle cells, cardiac cells, stem cells, fibroblasts, keratinocytes or cells derived from established cell lines, such as for example, 293, MDCK and C2C12 cell lines.


SEQUENCE LISTING (5 sequences)

SEQ ID NO: 1 - protein, 5 aa, Artificial Sequence
  Cell Adhesion Peptide 1
YIGSR

SEQ ID NO: 2 - protein, 4 aa, Artificial Sequence
  Cell Adhesion Peptide 2
REDV

SEQ ID NO: 3 - protein, 5 aa, Artificial Sequence
  Cell Adhesion Peptide 3
IKVAV

SEQ ID NO: 4 - protein, 10 aa, Artificial Sequence
  Cell Adhesion Peptide 4
KHIFSDDSSE

SEQ ID NO: 5 - protein, 4 aa, Artificial Sequence
  Cell Adhesion Peptide 5
KRSR